(19) United States
(12) Patent Application Publication
(10) Pub. No.: US 2024/0387047 A1
(43) Pub. Date: Nov. 21, 2024

(54) A METHOD AND SYSTEM DETECTING A HEALTH ABNORMALITY IN A LIQUID BIOPSY SAMPLE

(71) Applicant: OXFORD CANCER ANALYTICS (OXCAN), Oxford (GB)

(72) Inventors: Jian Rui LIU, Toronto (CA); Andreas HALNER, Oxford (GB)

(73) Assignee: OXFORD CANCER ANALYTICS (OXCAN), Oxford (GB)

(21) Appl. No.: 18/692,198

(22) PCT Filed: Sep. 15, 2022
(86) PCT No.: PCT/EP2022/075710

§ 371 Date: Mar. 14, 2024

Related U.S. Application Data
(60) Provisional application No. 63/244,293, filed on Sep. 15, 2021.

(51) Int. Cl. G16H 50/20

(52) U.S. Cl. CPC G16H 50/20

(57) ABSTRACT

The present disclosure relates to a computer implemented method for detecting a health abnormality in test data derived from a liquid biopsy. In obtaining the computer implemented method, special preparation and pre-processing of training and validation data for selecting relevant features and developing and selecting a well-performing final machine learning classifier to use for the final task of predicting the presence versus absence of a health abnormality. Finally, test data, which is obtained in-part from liquid biopsy samples, are input into the chosen machine learning classifier(s) and a screening or diagnostic test result of whether an associated patient has the health abnormality in question is provided.

### BACKGROUND

Medical screening and diagnostic testing on liquid biopsies is often used to detect the presence of a health abnormality, for example, cancer. Current cancer diagnostic tests almost exclusively focus on the ability to “see” a tumour via imaging and surgical resection is considered the curative treatment of choice in current standard of care. Unfortunately, such management in medical oncology is behind compared to how other diseases are managed. For example, guidelines indicate that ectopic pregnancy can be diagnosed by either the detection of a blood-based biomarker or ultrasound to “see” it. The presence of blood biomarkers above certain thresholds is sufficient to confirm ectopic pregnancy diagnosis and allow immediate treatment initiation. A recent proof of concept study, Chen, X. et al. (Non-invasive early detection of cancer four years before conventional diagnosis using a blood test. Nat Commun 11, 3475, doi: 10.1038/s41467-020-17316-z (2020)), has demonstrated that liquid biopsy is able to detect asymptomatic early cancer cases by up to 4 years before current gold standards.

### SUMMARY

As cancer management strategy and technologies such as liquid biopsy progress, a paradigm shift from physically “seeing” a cancer visually to confirm diagnosis to “seeing” a cancer based on more sensitive molecular level tools such as liquid biopsy will occur. Liquid biopsy has further potential to help determine the most suitable treatment modality for cancer patients using a molecular biomarker panel.

The present disclosure relates to a computer implemented method for detecting a health abnormality in test data derived from a liquid biopsy. In obtaining the computer implemented method, training data for a machine learning classifier model is established. Thereafter, a machine learning classifier(s), from a plurality of machine learning classifier(s) is chosen for detecting the health abnormality. Finally, test data, which is obtained in-part from liquid biopsy samples, are input into the chosen machine learning classifier(s) and a screening or diagnostic test result of whether an associated patient has the health abnormality in question is provided.

Thus, example embodiments described herein are directed towards establishing training data for a machine learning classifier model for use in detecting a health abnormality in a liquid biopsy sample. The method comprises receiving a plurality of data sets, where each data set comprises a plurality of features associated with a respective patient. The method further comprises identifying all m training data sets associated with a positive detection of the health abnormality and performing kernel density estimation on these data sets, followed by the creation of new ‘synthetic’ data sets consisting of p samples drawn at random from the positive health abnormality kernel density model. In addition, all n training data sets associated with an absence of the health abnormality are identified, kernel density estimation performed on these data sets and new ‘synthetic’ datasets are created consisting of q samples drawn at random from the absent health abnormality kernel density model. In this method, p≥m, q≥n and the values of p and q as well as the ratio p:q are parameters whose values are chosen based on clinical context and optimal performance of the classifier model in detecting the health abnormality in the validation data. The method also comprises compiling training data comprising both the p samples drawn at random from the positive health abnormality kernel density model and the q samples drawn at random from the absent health abnormality kernel density model. The method additionally comprises identifying relevant features within respective synthetic data sets of the training data, wherein a relevant feature, or combination of such features, provides a level of likelihood, above a threshold, of a positive indication of the health abnormality. The method also comprises optimizing training data via a removal of nonrelevant features. According to a further example embodiment of the present disclosure the method may further comprise retraining the classifier on a synthetic dataset (based on a combination of training and validation data) using an optimised synthetic dataset values of p and q and associated feature subset as determined by performance of the classifier in validation data. The classifier retrained in such a way can then be used to determine whether a patient in a test set has a health abnormality. Alternatively, a further example embodiment of the present disclosure may comprise retraining the classifier on original training and validation data, using any features identified as relevant during the validation stage using either original datasets or any one of the synthetic dataset methods (different values of p and q). In the latter embodiment, the machine learning classifier may select features identified as being particularly significant for distinguishing between patients with and without the health abnormality in the context of dataset imbalance which may occur in certain clinical situations. In some embodiments, this feature selection embodiment may increase the robustness of the performance of the machine learning classifier even if dataset imbalance occurs and/or if the signal associated with the health abnormality or the absence of the health abnormality is weak. The classifier retrained in such a way with the selected features can then be used to decide whether a patient in the test set has a given health abnormality.

Some of the example embodiments are directed towards a computer implemented method for selecting the machine learning classifier model for use in detecting the health abnormality in the liquid biopsy sample. The method comprises training a plurality of different machine learning classifier models using the optimized training data as described herein.

Some of the example embodiments are directed towards a computer implemented method for detecting the health abnormality in the liquid biopsy sample. The method comprises receiving a test data set comprising the identified relevant features as described herein, where the test data set is not equivalent to any data set comprised in the optimized training data set, and wherein the test data set comprises data corresponding to at least one liquid biopsy sample. The method further comprises assessing a performance of the selected machine learning classifier(s) on the test data set. The method additionally comprises receiving an output of the selected machine learning classifier(s), wherein the output indicates a presence of the health abnormality in the liquid biopsy sample corresponding to the test data set.

In computer implemented methods of the present disclosure, p≥m, q≥n and the values of p and q as well as the ratio p:q are parameters whose values are initialized based on clinical context and then further optimized via assessing performance of the classifier model in detecting the health abnormality in the validation data. The creation of synthetic data sets in this embodiment may be used to increase the total number of data records in the data set for training the classifier model to detect the health abnormality while keeping the ratio p:q of health abnormality versus absence of health abnormality synthetic data sets the same as the ratio of the original data records m:n. Alternatively, the creation of synthetic data sets in this embodiment may be used to modify the ratio p:q of health abnormality versus absence of health abnormality synthetic data sets as compared to the ratio of the original data records m:n. For example, the synthetic data sets may provide a skew in the distribution of patients which have and do not have the health abnormality such that either the representation of the health abnormality signal is amplified or the representation of the healthy (no health abnormality) signal is amplified, as desired. For example, according to some of the example embodiments, if the signal to noise ratio for the health abnormality in the multivariate feature space is strong and the number m of data records positive for the health abnormality is high but the number n of data records without the health abnormality is low, then the ratio of synthetic data sets p:q may be chosen such that (p/m)<(q/n). For example, in the former case if m=500 and n=200, p can be initialised at 750 and q can be initialised at 800 such that the number of synthetic data sets is greater than the number of original training data records and the density of the healthy (no health abnormality) synthetic data points in multivariate space compared to the density of the health abnormality synthetic data points in multivariate space has been increased relative to the density ratio in the original data records. This ensures that the classifier model has sufficient training data for the healthy (no health abnormality) samples. In another example embodiment where the number of original data records positive for the health abnormality is low compared to the number of original data records without the health abnormality and/or if the signal to noise ratio for the health abnormality in multivariate space is weak, then the ratio of synthetic data sets p:q may be chosen such that (p/m)>(q/n) with p<q or p=q or p>q. For example, in the case that m=200 and n=500, p may be initialised at 400 and q initialised at 500 or p may be initialised at 1,000 and q at 750. In the former p<q like m<n whereas in the latter case p>q although m<n but in both cases (p/m)>(q/n), so that the density of the healthy abnormality synthetic data points in multivariate space compared to the density of the healthy (no health abnormality) synthetic data points in multivariate space has been increased relative to the density ratio in the original data records. This ensures that the classifier model has sufficient training data for the positive health abnormality samples. After initialisation values of p and q based on contextual considerations including but not limited to the above, the exact values of the parameter p, q and of the ratio p:q are to be optimised by assessing the performance of the classifier model in the validation set. The creation of synthetic data sets from the positive health abnormality kernel density model and the absence of the health abnormality kernel density model rather than undersampling or oversampling with replacement enables a large number of synthetic data sets to be created while ensuring the presence of realistic noise amongst synthetic samples so that overfitting of the classifier is minimised when distinguishing between patients with versus patients without the health abnormality. A final feature subset may be determined by performance of the machine learning classifier in validation data using the particular synthetic data set with optimal values of p and q based on performance during the validation stage. Alternatively, the final feature set may include any features selected as relevant during the validation stage using either original datasets or any one of the synthetic dataset methods (different values of p and q). In the latter approach, the machine learning classifier may select features identified as being particularly important for distinguishing between patients with and without the health abnormality in the context of specified forms of dataset imbalance which may occur in certain clinical situations. In certain contexts, this feature selection approach can increase the robustness of the performance of the machine learning classifier even if dataset imbalance occurs and/or if the signal associated with the health abnormality or the absence of the health abnormality is weak. The classifier retrained in such a way with the selected features can then be used to decide whether a patient in the test set has a given health abnormality.

It should be appreciated examples of DNA may include mutations, copy number alterations, rearrangements, and circulating tumor DNA fragmentation size. Examples of proteomics may include both representations of quantity of protein as well as the presence of particular post-translational modifications of the protein. Protein data may be based on both mass spectrometry-based techniques with vary levels of depletion applied to the liquid biopsy as well as ELISA or other immune-assay based techniques for identifying particular proteins and protein forms in the liquid biopsy where such proteins and/or protein forms are already known to be or predicted by mathematical biological models to be up/down/regulated in tissues with the health abnormality compared to healthy tissue. Examples of epigenetics may include methylation, acetylation, and chromatin modifications. Further examples include quantitative and qualitative measures of bacterial or viral species present in liquid biopsy samples where such species are known to be associated or disassociated with a higher susceptibility to developing specific health abnormalities (e.g. malignancies). It should be appreciated the use of volatile organic molecules may be useful for breath analysis where the liquid biopsy is a breath sample.

According to some of the example embodiments, the analysis of biological features in the received data set is performed. The analysis may be both in the form of free molecules found in blood, urine, fecal matter, breath, sputum, etc. as well as the material obtained from tumor cells in the blood, urine, fecal matter, breath, sputum etc. as well as from exosomes in blood, urine, fecal matter, breath spectrum, etc.

According to some of the example embodiments, the linear dimensionality reduction may be (but is not limited to) a Principal Component Analysis (PCA) on the synthetic data sets (e.g. where the relevant features are features of the synthetic data set which yield highest variants in the PCA).

According to some of the example embodiments, a minimum predetermined metric may comprise at least one of area under the receiver operating characteristic curve, balanced accuracy, sensitivity, and specificity. An average decrease in classifier performance may be determined using a metric such as but not limited balanced accuracy. A response variable may comprise the presence or absence of a health abnormality.

According to some example embodiments, the learning classifier system may be a Michigan-style supervised learning classifier system or a Pittsburgh-style supervised learning system.

The threshold percentage is a highest percent of the plurality of machine learning classifier models or a sufficiently high percentage for clinical use performance metrics (such metrics may include but are not limited to area under the receiver operating characteristic curve, sensitivity, specificity, positive predictive value, and negative predictive value) of correctly detected health abnormalities.

According to some example embodiments, a method of assessing the performance of the selected machine learning classifier may be performed in relation to any computer implemented method of the present disclosure.

According to some example embodiments, a classification decision may be made based on a vote from some combination of any of the aforementioned classifiers.

In the case of a Michigan-style supervised learning classifier system or a Pittsburgh-style supervised learning system, as part of ‘expert knowledge discovery’ learning may also be guided by expert-based scores calculated from the extent to which elements of the received data set (elements from claim 3 from any of the liquid biopsy-contained materials in claim 4) are increased or decreased in liquid biopsy samples from patients with the health abnormality compared to liquid biopsy samples from patients without the health abnormality or in tissue biopsy samples from patients with the health abnormality compared to tissue biopsy samples from patients without the health abnormality or based on theoretical mathematical biological predictions of an increase or decrease in quantity of a certain element in a state of health abnormality versus absence of the health abnormality.

### BRIEF DESCRIPTION OF THE DRAWINGS

FIG. 1 is an illustrative overview of how data sets are used according to some of the example embodiments described herein;

FIG. 2 is an illustrative example of a data record, according to some of the example embodiments described herein;

FIG. 3 is an illustration of an apparatus for establishing training data, selecting a machine learning classifier model and detecting a health abnormality in a liquid biopsy, according to some of the example embodiments described herein;

FIG. 4 is a flow chart of example operations for establishing training data for a machine learning classifier model for use in detecting a health abnormality in a liquid biopsy sample, according to some of the example embodiments described herein;

FIG. 5 is a flow chart of example operations for selecting the machine learning classifier model for use in detecting the health abnormality in the liquid biopsy sample, according to some of the example embodiments described herein; and

FIG. 6 is a flow chart of example operations detecting the health abnormality in the liquid biopsy sample, according to some of the example embodiments described herein.

FIG. 7 is a table illustrating the number of KDE-based samples used during training for each cancer type using Cohen et al. (“Detection and localization of surgically resectable cancers with a multi-analyte blood test.” Science. 2018 Feb. 23; 359 (6378): 926-930. doi: 10.1126/science.aar3247) dataset.

FIG. 8 is a table illustrating the number of proteins selected using original data, KDE methods and the final union feature set.

FIG. 9 is a table illustrating test set sensitivity of random forest model using 28 protein union feature set according to cancer type and stage for an overall specificity threshold of 99%.

FIG. 10 is a graph illustrating test set receiver operating characteristic curve and area under receiver operating characteristic curve (AUC) showing the performance of the example embodiments for distinguishing 201 cancer patients from 163 cancer free patients of the Cohen et al dataset.

FIG. 11 is a table illustrating cross-validation sensitivity reported by Cohen et al according to cancer type and stage for an overall specificity threshold of 99%.

FIG. 12 is a table illustrating the number of KDE-based samples used during training for lung cancer versus cancer free classification using Blume et al (“Rapid, deep and precise profiling of the plasma proteome with multi-nanoparticle protein corona.” Nat Commun. 2020 Jul. 22; 11 (1): 3662. doi: 10.1038/s41467-020-17033-7) dataset.

FIG. 13 is a table illustrating the number of proteins selected using original data, KDE methods and the final union feature set.

FIGS. 14A TO 14F are a series of graphs illustrating receiver operating characteristic curves and area under receiver operating characteristic curve (AUC) showing the 31-patient test set performance of the optimised random forest classifier model for each spion or depleted plasma data of Blume et al.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the invention. Instead, they are merely examples of apparatuses and methods consistent with aspects related to the invention as recited in the appended claims.

The disclosed embodiments relate to methods and systems for applying machine learning techniques for diagnostic methods of testing for health abnormalities. Example embodiments described herein are directed towards establishing a system to provide screening or diagnostic test results on test data derived, in-part, from liquid biopsies. In establishing the screening or diagnostic system, some of the example embodiments are directed towards training data being generated and optimized. Thereafter, the example embodiments further comprise a means of selecting a machine learning classifier(s), from a plurality of possible machine learning classifier(s), for providing the screening or diagnostic test. Finally, some of the example embodiments described herein are directed towards providing a screening or diagnostic test result on test data, obtained in part from a liquid biopsy, associated with a particular patient. The screening or diagnosis described herein is an indication as to whether the associated patient has the health abnormality in question.

The example embodiments are described herein using lung cancer as an example health abnormality. However, it should be appreciated the example embodiments described herein may be applied to any other form of cancer as well as autoimmune diseases and neurodegenerative conditions. It should further be appreciated a liquid biopsy as described herein may comprise a sample of a sample of blood, urine, fecal matter, breath, or sputum.

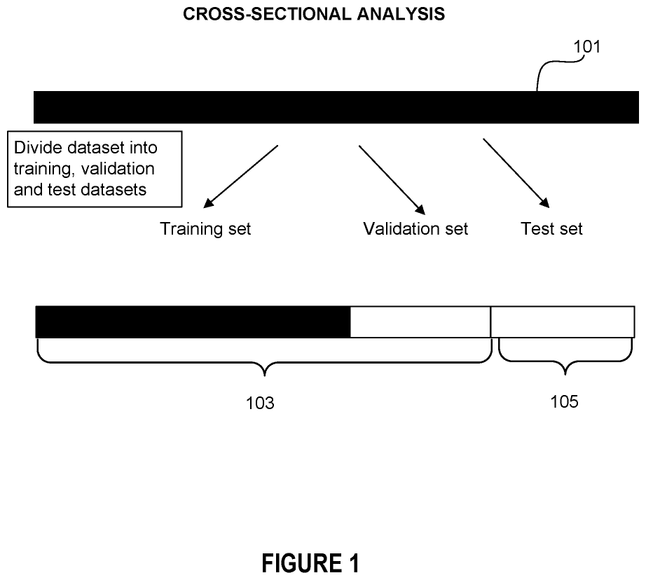

In determining the health abnormality from the liquid biopsy sample and establishing a system for the same, machine learning techniques using various forms of data may be employed. FIG. 1 provides an overview of how data is utilized, according to some of the example embodiments described herein. Various data sets 101 may be used, for example a training data set, a validation data set and a test data set. Each data set may comprise any number of data records and each data record may comprise a plurality of features. The training and validations data sets 103 may be utilized in choosing and optimization a machine learning classifier(s) for providing the diagnostic.

The training data set comprises data records featuring a number of known positive cases or data associated with patients which are known to have the health abnormality. The training data set further comprises a number of data records associated with healthy patients or patients whose data would yield a negative result for the health abnormality in question. According to some of the example embodiments, kernel density estimation is performed and synthetic data sets are created by drawing samples from the positive detection of the health abnormality kernel density model and from the absence of health abnormality kernel density model, such that the synthetic data sets may comprise of a different number of cases with and without the health abnormality as well as a different ratio of cases with versus without the health abnormality as compared to the original data records.

The validation data set is used to select a machine classifier model. The validation data set is further used to optimise the number of synthetic data sets from the positive detection of the health abnormality kernel density model versus the number of synthetic data sets from the absence of health abnormality kernel density model. The validation data set is further used to adjust parameters of the chosen machine classifier model in order to provide a desired metric, for example, a particular sensitivity and specificity ratio. Test data sets comprise the data or patients to be tested in with the finalized machine classier model.

Training and validation and data sets 103 may be two separate sets as shown or instead k-fold cross-validation may be used wherein the ‘test set’ is first separated out from the ‘total data’ and the rest of the data is divided into different train-validation splits.

The final machine classifier models (type of classifier(s) and particular feature combinations) determined based on performance on the ‘validation set’ or ‘validation folds’ are now assessed on the ‘test set’ which has hitherto not been used in any way as part of the training and validation steps.

Testing data sets 105 are the data sets to be tested using the selected and optimized machine classifier model. The testing data set 105 is formatted in a similar manner as the training data sets and the validation data sets 103. The machine classifier model will provide an analysis as to whether the patient associated with the testing data set 105 has the health abnormality in question.

It should be appreciated a single machine learning classifier may be chosen or a combination of machine learning classifiers may be employed. Once the machine learning system is established, test data 105, which is associated with a particular patient, is input into the chosen machine learning classifier(s) and a resulting decision regarding the presence or absence of the health abnormality will be provided as an output.

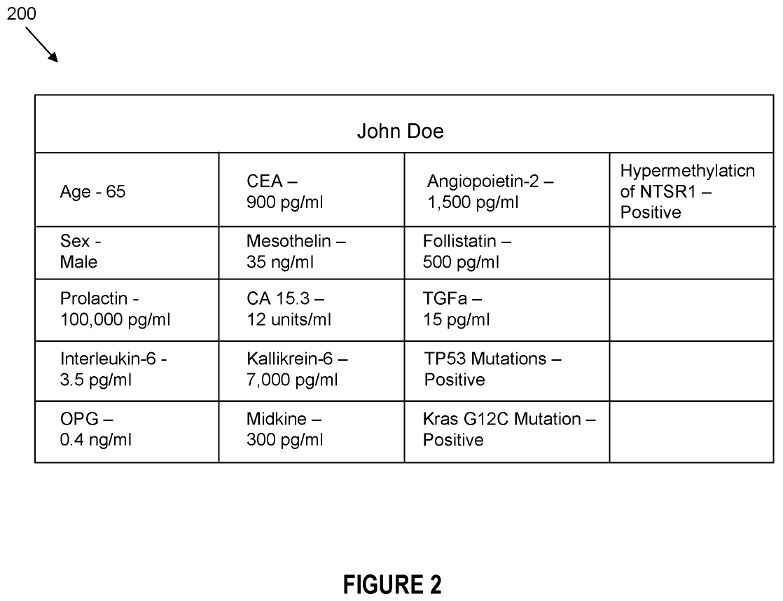

FIG. 2 provides an illustrative example of a data record 200. Training data, validation data and test data may all take the form of the example shown in FIG. 2. According to some of the example embodiments, the data record may comprise information specific to a particular patient. In the example provided by FIG. 2, the patient is John Doe. The data record may comprise various features or variables which are associated with John Doe. For example, two features illustrated in the data record 200 are John Doe's age and gender, 65 and male, respectively.

Other example variables or features include circulating proteins (e.g. prolactin, interleukin-6, OPG, CEA, mesothelin, CA 15-3, kallikrein-6, midkine, angiopoietin-6, follistatin and TGFa) which are present in the liquid biopsy sample or are proteins produced by the immune system in response to the abnormal tissue. In addition, variables may include the presence and/or amount of circulating DNA fragments in the liquid biopsy sample where such DNA fragments have mutations (e.g. mutations of the gene TP53, a gene normally involved in suppressing tumour development) associated with the health abnormality which are present in the liquid biopsy sample. Variables may also include the presence and/or quantity of epigenetic DNA modifications which regulate gene expression. Methylation is one example of such an ‘epigenetic’ change which can be associated with certain disease states (e.g. the presence of hyper-methylation of the NTSR1 gene).

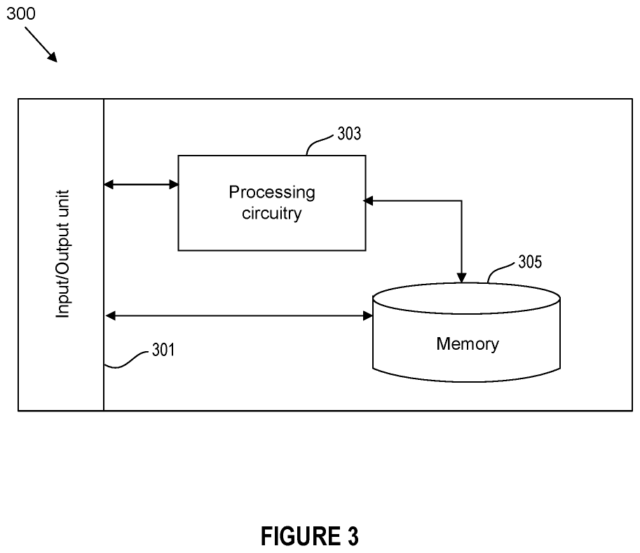

FIG. 3 is an example hardware configuration of an analysis unit 300 configured to generate training data, select and optimize a machine learning classifier(s) and provide diagnostic testing as described herein. The unit 300 may comprise an input/output unit 301 that is configured to receive and/or transmit data, instructions or messages. It should be appreciated the input/output unit 301 may be in the form of any input or output communications port known in the art. The unit 300 may further comprise processing circuitry 303. The processing circuitry 303 may be any suitable type of computation unit, for example, a microprocessor, digital signal processor (DSP), field programmable gate array (FPGA), or application specific integrated circuitry (ASIC), or any other form of circuitry. The analysis unit 300 may further comprise a memory unit 305 which may be any suitable type of computer readable memory and may be of volatile and/or non-volatile type. The memory 303 may be configured to store received, transmitted, and/or measured data and/or executable program instructions.

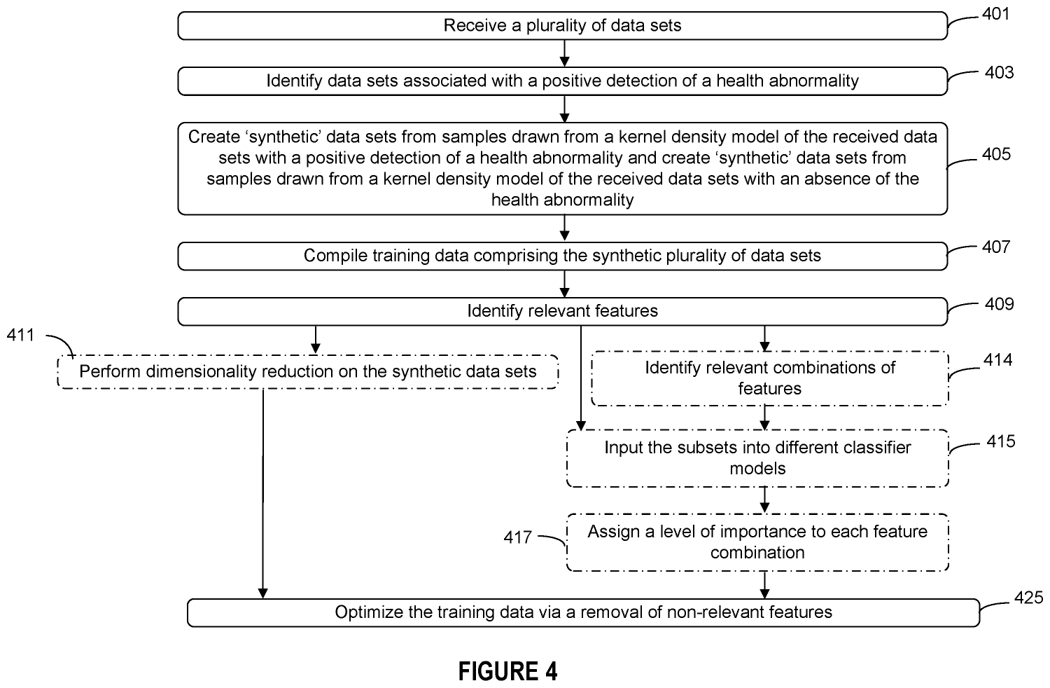

FIG. 4 is a flow diagram depicting example operations which may be taken by the unit 300 as described herein in establishing training data for a machine classifier model. It should be appreciated that FIG. 4 comprises some operations which are illustrated with a solid border and some operations which are illustrated with a dashed border. The operations which are comprised in a solid border are operations which are comprised in the broadest example embodiments. The operations which are comprised in the dashed border are example embodiments which may be comprised in, or a part of, or are further operations which may be taken in addition to the operations of the boarder example embodiments. It should also be appreciated that the actions may be performed in any order and any combination.

### Operation 401

The example embodiments comprise receiving a plurality of data sets, each data set comprising a plurality of features associated with a respective patient. The input/output unit 301 is configured to receive the plurality of data sets.

According to some of the example embodiments, the data set may be formatted as depicted in FIG. 2. As this data is used to establish the training data for the machine classifier model, it is known whether or not the patients associated with respective data sets comprise the health abnormality. The data sets are associated with a liquid biopsy sample. According to some of the example embodiments, the liquid biopsy sample may be in the form of blood, urine, fecal matter, breath or spectrum.

According to some of the example embodiments, the received data set comprises any one or more of DNA, epidemiology based data, proteomics, epigenetics, volatile organic molecules, metabolomics and/or microbiome based data. It should be appreciated examples of DNA may include mutations, copy number alterations, rearrangements, and circulating tumour DNA fragmentation size. Examples of proteomics may include both representations of quantity of protein as well as the presence and quantity of particular post-translational modifications of the protein. Protein data may be based on both mass spectrometry-based techniques with varying levels of depletion applied to the liquid biopsy as well as ELISA or other immune-assay based techniques for identifying particular proteins and protein forms in the liquid biopsy where such proteins and/or protein forms are already known to be up/down/regulated in tissues with the health abnormality compared to healthy tissue. Examples of epigenetics may include methylation, acetylation and chromatin modifications. Further examples include quantitative and qualitative measures of bacterial or viral species present in liquid biopsy samples where such species are known to be associated or disassociated with a higher susceptibility to developing specific health abnormalities (e.g. malignancies). It should be appreciated the use of volatile organic molecules may be useful for breath analysis where the liquid biopsy is a breath sample.

According to some of the example embodiments, the analysis of biological features in the received data set is performed. The analysis may be both in the form of free molecules found in blood, urine, fecal matter, breath, sputum, etc. as well as the material obtained from tumour cells in the blood, urine, fecal matter, breath sputum etc. as well as form exosomes in blood, urine, fecal matter, breath spectrum, etc.

### Operation 403

The example embodiments further comprise identifying data sets, of the received data sets, associated with a positive detection of a health abnormality. The processing circuitry 303 is configured to identify the data sets associated with the positive detection of the health abnormality. As described previously, the obtained data, used to compile the training data, features information from known patients and therefore patients which have the health abnormality are known beforehand.

### Operation 405

The example embodiments further comprise identifying all m training data sets associated with a positive detection of the health abnormality and performing kernel density estimation on these data sets, followed by the creation of first ‘synthetic’ data sets consisting of p samples drawn at random from the positive health abnormality kernel density model. In addition, all n training data sets associated with an absence of the health abnormality are identified, kernel density estimation performed on these data sets and second ‘synthetic’ datasets are created consisting of q samples drawn at random from the absent health abnormality kernel density model. The processing circuitry 303 is configured to perform kernel density estimation and draw samples from the kernel density models as hitherto described, resulting in the plurality of synthetic data sets.

According to some of the example embodiments, p>m and/or q>n and/or the ratio p:q is different from the ratio m:n. The values of p and q as well as the ratio p:q are parameters whose values are initialised based on clinical context and then further optimised via assessing performance of the classifier model in detecting the health abnormality in the validation data. The creation of synthetic data sets in this embodiment may be used to increase the total number of data records in the data set for training the classifier model to detect the health abnormality while keeping the ratio p:q of health abnormality versus absence of health abnormality synthetic data sets the same as the ratio of the original data records m:n. Alternatively, the creation of synthetic data sets in this embodiment may be used to modify the ratio p:q of health abnormality versus absence of health abnormality synthetic data sets as compared to the ratio of the original data records m:n. For example, the synthetic data sets may provide a skew in the distribution of patients which have and do not have the health abnormality such that either the representation of the health abnormality signal is amplified or the representation of the healthy (no health abnormality) signal is amplified, as desired. For example, according to some of the example embodiments, if the signal to noise ratio for the health abnormality in the multivariate feature space is strong and the number m of data records positive for the health abnormality is high but the number n of data records without the health abnormality is low, then the ratio of synthetic data sets p:q may be chosen such that (p/m)< (q/n). For example, in the former case if m=500 and n=200, p can be initialised at 750 and q can be initialised at 800 such that the number of synthetic data sets is greater than the number of original training data records and the density of the healthy (no health abnormality) synthetic data points in multivariate space compared to the density of the health abnormality synthetic data points in multivariate space has been increased relative to the density ratio in the original data records. This ensures that the classifier model has sufficient training data for the healthy (no health abnormality) samples. In another example embodiment where the number of original data records positive for the health abnormality is low compared to the number of original data records without the health abnormality and/or if the signal to noise ratio for the health abnormality in multivariate space is weak, then the ratio of synthetic data sets p:q may be chosen such that (p/m)> (q/n) with p<q or p=q or p>q. For example, in the case that m=200 and n=500, p may be initialised at 400 and q initialised at 500 or p may be initialised at 1,000 and q at 750. In the former p<q like m<n whereas in the latter case p>q although m<n but in both cases (p/m)> (q/n), so that the density of the healthy abnormality synthetic data points in multivariate space compared to the density of the healthy (no health abnormality) synthetic data points in multivariate space has been increased relative to the density ratio in the original data records. This ensures that the classifier model has sufficient training data for the positive health abnormality samples. After initialisation values of p and q based on contextual considerations including but not limited to the above, the exact values of the parameter p, q and of the ratio p:q are to be optimised by assessing the performance of the classifier model using a range of different values for p, q and p:q in the validation set and selecting p, q and p:q corresponding to the classifier model which performs best in the validation set. The creation of synthetic data sets from the positive health abnormality kernel density model and the absence of the health abnormality kernel density model rather than undersampling or oversampling with replacement enables a large number of synthetic data sets to be created which ensure the presence of realistic noise amongst synthetic samples so that overfitting of the classifier is minimised when distinguishing between patients with versus patients without the health abnormality.

According to some of the example embodiments, the final feature subset as determined by performance of the machine learning classifier in validation data using the particular synthetic data with optimal values of p and q based on performance during the validation stage. Alternatively, the final feature set may include any features selected as relevant during the validation stage using either original datasets or any one of the synthetic dataset methods (different values of p and q). In the latter approach, the machine learning classifier may select features identified as being particularly important for distinguishing between patients with and without the health abnormality in the context of dataset imbalance which may occur in certain clinical situations. In certain contexts, this feature selection approach can increase the robustness of the performance of the machine learning classifier even if dataset imbalance occurs and/or if the signal associated with the health abnormality or the absence of the health abnormality is weak. The classifier retrained in such a way with the selected features can then be used to decide whether a patient in the test set has a given health abnormality.

### Operation 407

Example embodiments further comprise compiling training data comprising the plurality synthetic of data sets. The processing circuitry 303 is configured to compile the training data comprising the plurality of synthetic data sets. Thus, the training data used herein comprises of synthetic data sets wherein data sets related to kernel density models for patients which have the health abnormality are more or less heavily used compared to data sets related to patients without the health abnormality compared to the original data records, depending on the synthetic data set health abnormality: no health abnormality ratio optimal for the classifier.

### Operation 409

The Example embodiments further comprise identifying relevant features within respective synthetic data sets of the training data, wherein a relevant feature, or combination of features, provides a level of likelihood, above a threshold, of a positive indication of the health abnormality.

As illustrated in FIG. 2, each data record will comprise a number of features related to a respective patient. In order to optimize the process of determining whether or not the respective patient has the health abnormality, features which are deemed not to be relevant may be removed from the data record in order to reduce the required processing need per data record.

### Example Operation 411

According to some of the example embodiments, the identifying of relevant features (operation 409) may comprise performing a linear dimensionality reduction such as Principal Component Analysis (PCA) on the synthetic data sets (e.g. where the relevant features are features of the synthetic data set which yield highest variants in the PCA) or non-linear dimensionality reduction techniques. The processing circuitry 303 may be configured to perform the dimensionality reduction on the synthetic data sets.

### Example Operation 415

According to some of the example embodiments, the identifying of relevant features (operation 409) may comprise identifying the relevant features via inputting the complied training data into a variety of classifier models to identify non-linear feature interactions. The processing circuitry 303 may be configured to input the complied training data into a variety classifier models to identify non-linear feature interactions.

According to some of the example embodiments, randomized feature subsets containing x features (where x may take a different value for different subsets but in all cases x<the total number of features y) may first be compiled (example operation 414). These randomized subsets may be input into the variety of classifier models to identify the non-linear feature interactions. The use of such randomized subsets of x features may enable the testing of a greater number of feature combinations whilst preventing overfitting, especially if the total number of features y exceeds the total number of training data sets.

According to some of the example embodiments, the plurality of different classifier models comprise a learning classifier system. The learning classifier system may be a Michigan-style supervised learning classifier system or a Pittsburgh-style supervised learning system.

### Example Operation 417

According to some of the example embodiments, upon compiling the randomized subsets (example operation 413) and/or inputting the subsets into different classifier models (example operation 415), the identifying of relevant features (operation 409) may further comprise selection of feature subsets which enable the classifier to yield a minimum predetermined metric (e.g. balanced accuracy, sensitivity, specificity) in the validation set or else the selection of a proportion of top-performing classifier feature subsets. Next, different combinations of features (number of features in a combination may range from 1 to x) which occur in at least a specified proportion of selected feature subsets is noted and the total number of such combinations z is noted. A level of importance is then assigned to each of the z feature combinations. The processing circuitry 303 may be configured to assign the level of importance to each of the z feature combinations, for example, based on the average decrease in classifier performance (using a metric such as but not limited to balanced accuracy) across all feature subsets containing the particular feature combination when in the relevant feature subsets all of the features of the particular feature combination are deleted or the features' values permuted in relation to the response variable (presence or absence of health abnormality) such that the features of a particular combination are rendered non-informative. Hence, a level of importance (for enabling the classifier to distinguish between the presence and absence of the health abnormality) is assigned not only to individual features but also to particular combinations of features which are part of at least a specified proportion of selected feature subsets.

### Operation 425

The example embodiments further comprise optimization training data via a removal of nonrelevant features. The processing circuitry 303 is configured to optimize the training data via a removal of nonrelevant features.

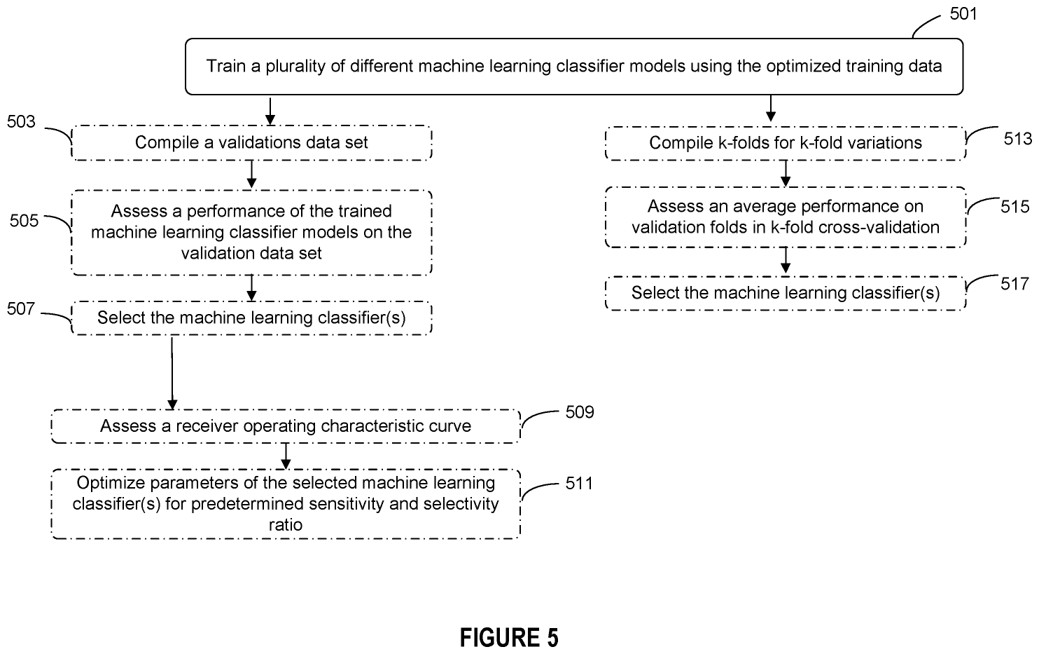

FIG. 5 illustrates example operations which may be performed by the analyzing unit 300 in selecting and training machine learning classifier models using the optimized training data described above. It should be appreciated that FIG. 5 comprises some operations which are illustrated with a solid border and some operations which are illustrated with a dashed border. The operations which are comprised in a solid border are operations which are comprised in the broadest example embodiments. The operations which are comprised in the dashed border are example embodiments which may be comprised in, or a part of, or are further operations which may be taken in addition to the operations of the boarder example embodiments. It should also be appreciated that the actions may be performed in any order and any combination.

### Operation 501

The example embodiments comprise training a plurality of different machine learning classifier models using the optimized training data described above. The processing circuitry 303 is configured to train the plurality of different machine learning classifier models using the optimized training data described above.

### Example Operation 503

According to some of the example embodiments, the training (operation 501) further comprises compiling a validation data set, wherein the validation data set comprises the identified relevant features and wherein the validation data set is not equivalent to any data set comprised in the optimized data set. The processing circuitry 303 may be configured to compile the validation set. As described in FIG. 1, the validation data set is distinct from the training data and may be used to further optimize the classifier model. It should be appreciated the validation data set may comprise the same form as the example provided in FIG. 2.

### Example Operation 505

Some of the example embodiments may further comprise assessing a performance of the trained machine learning classifier models on the validation data set. The processing circuitry 303 may be configured to access the performance of the trained machine learning classifier model on the validation data set.

It should be appreciated that as is the case with the training data, the data records of the validation data set are associated with known patients. Therefore, whether the patient is affected by the health abnormality is known beforehand. Thus, the accuracy of the machine learning classifier models may be determined using the validation data set.

### Example Operation 507

Some of the example embodiments further comprise selecting the machine learning classifier(s), from a plurality of machine learning classifier models, wherein the selected machine learning classifier(s) yield a percentage above a threshold of correctly detected health abnormalities. The processing circuitry 303 may be configured to select the machine learning classifier(s) from the plurality of machine learning classifier models. According to some of the example embodiments, the machine learning classifier(s) which yield the highest percentage of correctly detected health abnormalities may be chosen.

### Example Operation 509

Some of the example embodiments may further comprise assess performance of the machine learning model on the validation data set via a receiver operating characteristic curve. The processing circuitry 303 is configured to assess the performance of the machine learning model on the validation set via the receiving operating characteristic curve.

### Example Operation 511

Some of the example embodiments further comprise optimizing parameters of the selected machine learning classifier(s) to obtain predetermined sensitivity and selectivity ratios on the receive operating characteristics curve. The processing circuitry 303 may be configured to optimize parameters of the selected machine learning classifier(s), for example, to obtain the predetermined sensitivity and selectivity ratios of the receiving operating characteristics curve. It should be appreciated that different ratios may be applied for different applications. For example, a threshold required for a certain clinical application (e.g. for the purposes of improving upon current standard of care and/or achieving cost-effectiveness within the context of a particular healthcare system) can be chosen. One example from our work is fixing specificity to 99% and identifying what sensitivity we can achieve while maintaining specificity at 99%. A contrasting example would be to instead maximise sensitivity at the expense of specificity while still ensuring that specificity does not drop below a certain threshold e.g. 80% specificity. A third approach is to simply maximise one of sensitivity or specificity at the expense of the other.

### Example Operation 513

Some of the example embodiments comprise compiling k-fold variations. The processing circuitry may be configured to compile the k-fold variations. The k-folds are k training-validation folds i.e. after the test set data are separated from the rest, the remaining data (103 in FIG. 1) are divided into train-validation splits. For example, in 5 fold cross validation, data set 103 in FIG. 1 would consist of 5 shuffles of 4/5 training 1/5 validation data. In 10 fold cross validation, data set 103 in FIG. 1 would consist of 10 shuffles of 9/10 training 1/10 validation data etc.

### Example Operation 515

Some of the example embodiments further comprise assessing an average performance on validations folds in the k-fold cross-validation. The processing circuitry 303 may be configured to assess the average performance on the validations fold in the k-fold cross-validation.

### Example Operation 517

Some of the example embodiments comprise selecting the machine learning classifier(s), from the plurality of machine learning classifier models, where the selected machine learning classifier(s) yield a percentage above a threshold of correctly detected health abnormalities. The processing circuitry 303 may be configured to select the machine learning classifier(s) from the plurality of machine learning classifier models. According to some of the example embodiments, the machine learning classifier(s) which yield the highest percentage of correctly detected health abnormalities may be chosen.

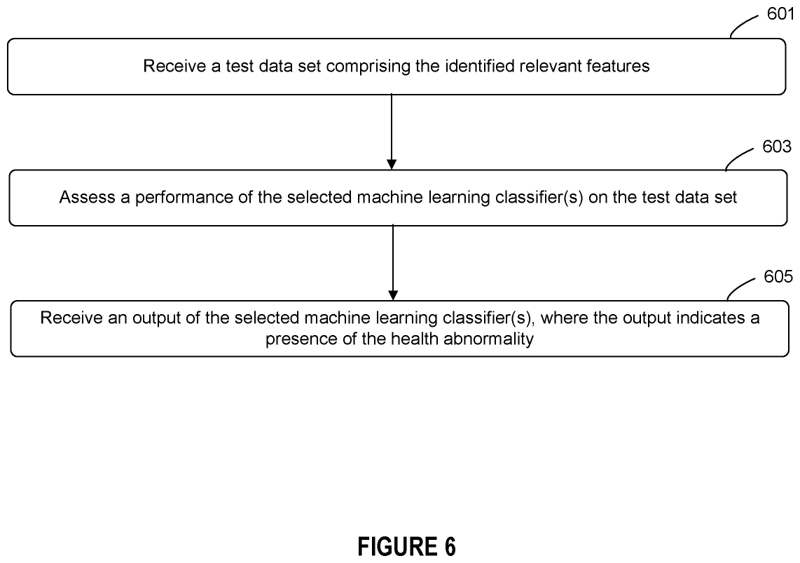

FIG. 6 illustrates example operations which may be performed by the analyzing unit 300 in detecting the health abnormality in a test data set using the selected machine learning classifier(s) described above.

### Operation 601

The embodiments comprise receiving a test data set comprising the identified relevant features as described above. The test data set is not equivalent to any data set comprised in the optimized training data set. The test data set comprises data corresponding to at least one liquid biopsy. The input/output unit 301 is configure receive the test data set comprising the identified relevant features described above. It should be appreciated the data records of the test data set may be of the same form as the example provided in FIG. 2. According to some of the example embodiments, the data records of the test data set may comprise the identified relevant features as described in relation to FIG. 4.

According to some of the example embodiments, the classifier model is a one or more of a support vector machine, neural network, decision tree, random forest, boosted tree, logistic regression, lasso, k-nearest neighbour, and/or naïve byes. It should be appreciated a classification decision may be made based on a vote from some combination of any of the aforementioned classifiers.

According to some of the example embodiments, the classifier model is a Michigan-style supervised learning classifier system or a Pittsburgh-style supervised learning system. In the case of a Michigan-style supervised learning classifier system or a Pittsburgh-style supervised learning system, as part of ‘expert knowledge discovery’ learning may also be guided by expert-based scores calculated from the extent to which elements of the received data set are increased or decreased in liquid biopsy samples from patients with the health abnormality compared to liquid biopsy samples from patients without the health abnormality or in tissue biopsy samples from patients with the health abnormality compared to tissue biopsy samples from patients without the health abnormality or based on theoretical mathematical biological predictions of an increase or decrease in quantity of a certain element in a state of health abnormality versus absence of the health abnormality.

### Operation 603

Example embodiments further comprise assessing a performance of the selected machine learning classifier(s) on the test data set as described above. The processing circuitry 303 is configured to assess the performance of the selected machine learning classifier(s).

### Operation 605

The example embodiments further comprise receiving an output of the selected machine learning classifier(s), where the output indicates a presence of the health abnormality in the liquid biopsy sample corresponding to the test data set. The processing circuitry 303 is configured to receive the output of the selected machine learning classifier(s), where the output indicates the presence of the health abnormality in the liquid biopsy sample corresponding to the test data set. According to some of the example embodiments, the output may be a probability or vote corresponding to a presence versus an absence of the health abnormality.

Advantages and benefits of embodiments of the present disclosure are illustrated by the following examples. In such following examples, computer implemented methods of the present disclosure are applied to the following two open access datasets:

First open access dataset: Cohen et al, a multi-cancer dataset including protein and DNA blood measurements. 1,005 patients had one of eight types of cancer (including breast, colorectum, esophagus, liver, lung, ovarian, pancreatic and stomach). 812 patients were cancer-free. The Cohen et al dataset enables the assessment of the ability of embodiments of the present disclosure to detect and distinguish multiple different cancer types. This reflects an example clinical context in which both cancer detection and localization are required. The number of cancer patients was as follows: lung, 104; breast, 209; colorectum, 388; oesophagus, 45; liver, 44; ovary, 54; pancreas, 93; stomach, 68.

Second open access dataset: Blume et al, a high dimensional proteomics dataset. 141 patients were included. 61 patients had lung cancer and 80 patients were cancer-free. The Blume et al dataset enables the assessment of the ability of the example embodiments to perform feature selection and use a small subset of the original dataset features to detect lung cancer.

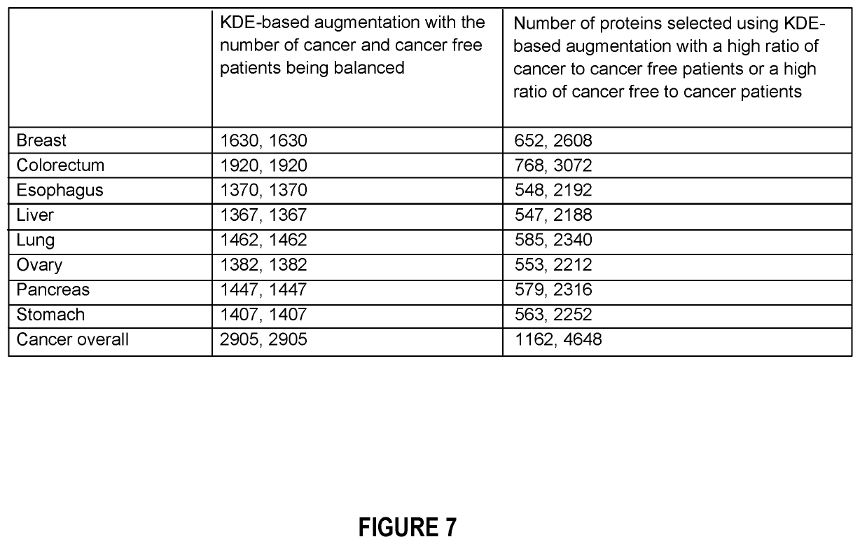

In this example, computer implemented methods of embodiments of the present disclosure were assessed on a hold-out test set of approximately 20% of samples to provide a generalisable estimate of performance. The test set was stratified according to cancer type and in the case of the Cohen et al dataset also in terms of stage in order to reflect all cancer types and the same proportion of stage 1, 2 and 3 cancers as occurred in the overall dataset. The remaining patient samples not part of the hold-out test set constituted data for training and validation of cancer detection classifiers in a Monte Carlo cross validation scheme. The validation includes feature selection steps and classifier optimization combined with various kernel density-based estimation (KDE) methods. In addition to using the original, untransformed data, three KDE-based methods were used: data augmentation with a balanced ratio of cancer and cancer free patients; data augmentation with a higher ratio of cancer to cancer free patients; data augmentation with a higher ratio of cancer free to cancer patients. The table of FIG. 7 shows the number of KDE-based samples used during training for each cancer type.

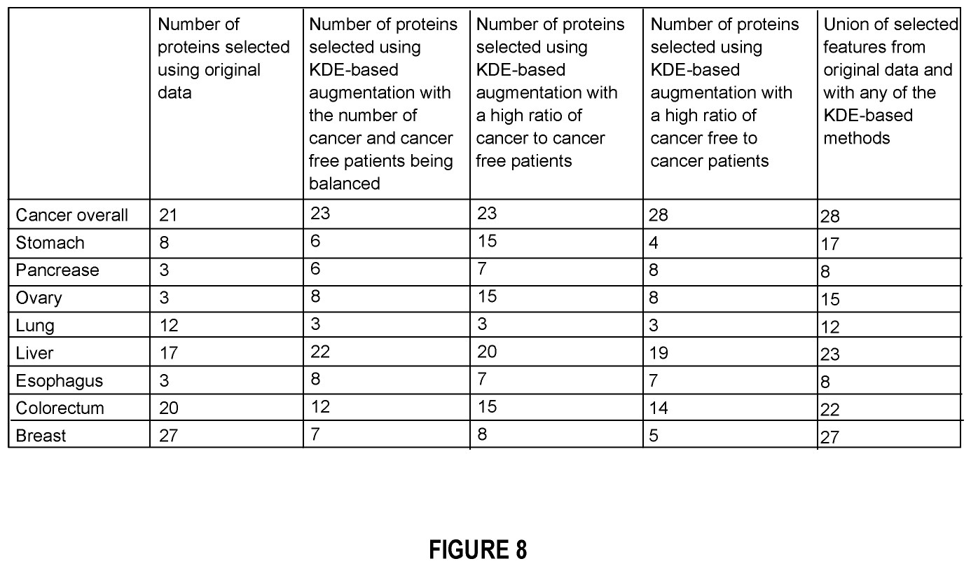

A random forest classifier was trained using all 39 proteins in Cohen et al. Recursive feature elimination was performed using the random forest Gini-index based variable importance score. The optimal subset of features was selected which yielded the statistically significantly highest classification area under the receiver operating characteristic curve of the different feature set size classifiers. Once the optimal protein subset was identified for a given KDE-based method and original data, hypothesis testing was performed to select the smallest subset of proteins which yielded an area under the receiver operating characteristic curve not statistically lower than that of the best performing protein subset. Finally, the union of the feature sets selected after hypothesis testing for original data and each of the three KDE-based methods was selected. Using this union feature set, hyperparameter optimisation was performed for random forest, support vector machine, logistic regression with 12 penalty and multilayer perceptron. The best performing classifier model with the union optimal protein feature subset was identified according to area under the receiver operating characteristic curve across the Monte Carlo cross validation folds. This optimal model was retrained on all training and validation patient samples and then assessed on the hold out test set. For the cancer versus cancer free classification, in addition to test set area under the receiver operating characteristic curve, the test set sensitivities by cancer and stage for an overall 99% specificity were reported. This enabled a comparison with the Cohen et al context in which minimising false positives was one of the objectives for a cancer detection algorithm. The table of FIG. 8 shows the number of proteins selected using original data as well as each of the three KDE-based methods and the number of proteins in the union feature set.

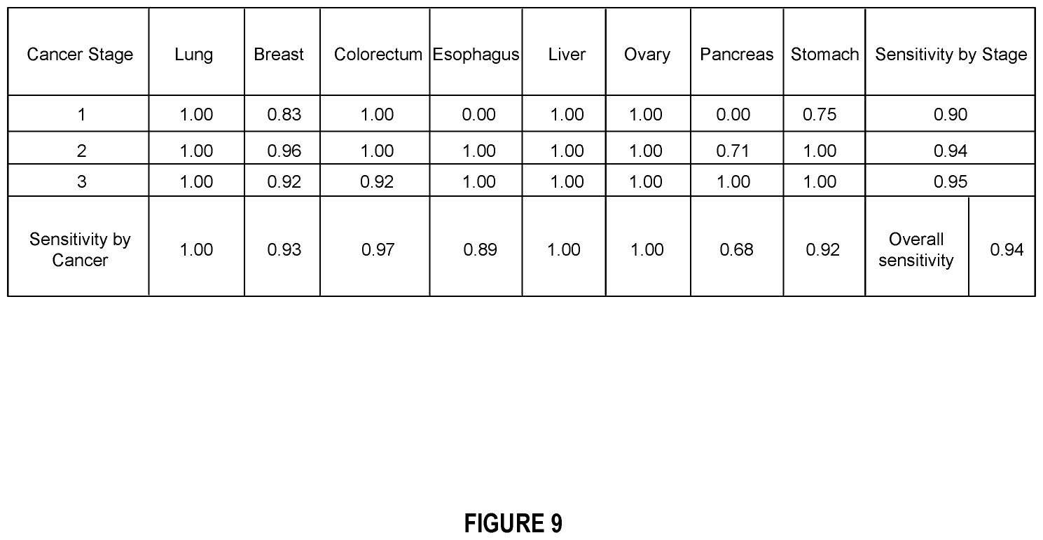

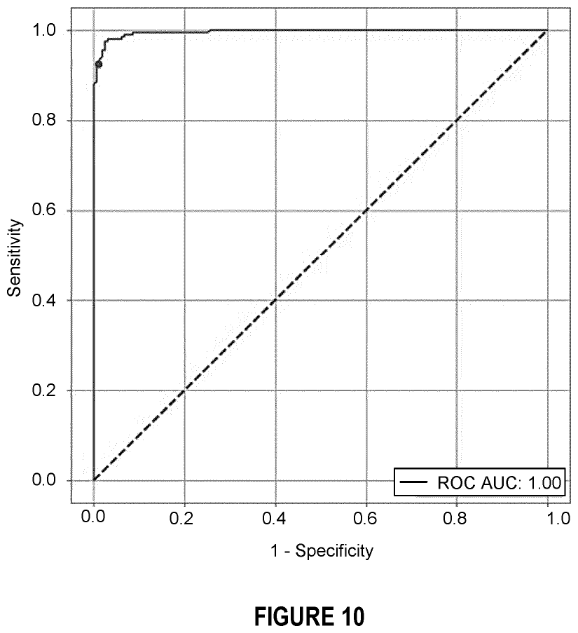

The performance of the example embodiments was superior to that of the Cohen et al paper. The performance of the example embodiments is summarised in the table of FIG. 9. For example, for distinguishing between cancer overall and cancer free patients, the machine learning framework of the example embodiments achieved an overall sensitivity for stage 1, 2 and 3 cancers are 90%, 94% and 95% respectively. For cancer type irrespective of stage, the sensitivity ranged from 68% in the case of pancreatic cancer to 100% for lung, liver, and ovarian cancer. FIG. 10 shows the receiver operating characteristic curve (ROC) of the machine learning pipeline of the example embodiments to distinguish overall cancer from cancer free patients.

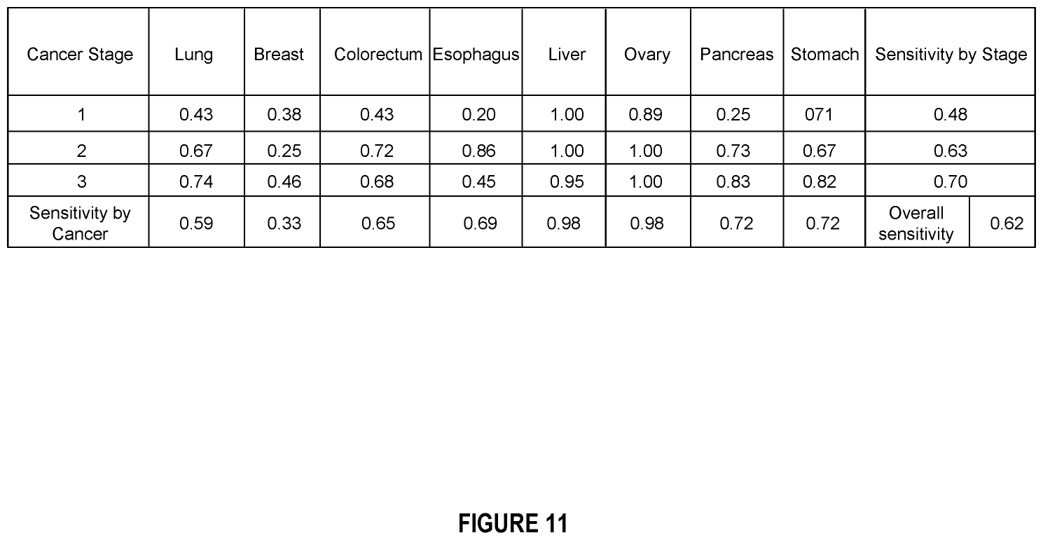

The performance of the example embodiment is greater than the performance of Cohen et al's approach of 48%, 63% and 70% sensitivity for stage 1, 2 and 3 respectively without the example embodiments. The performance of Cohen et al's approach not using the example embodiments is summarised in the table of FIG. 11. Notably, overall, across all stages the overall cancer versus cancer free example embodiments pipeline achieved a sensitivity of 100% for lung, 93% for breast and 97% for colorectal cancer compared to Cohen et al's 59%, 33% and 65% respectively.

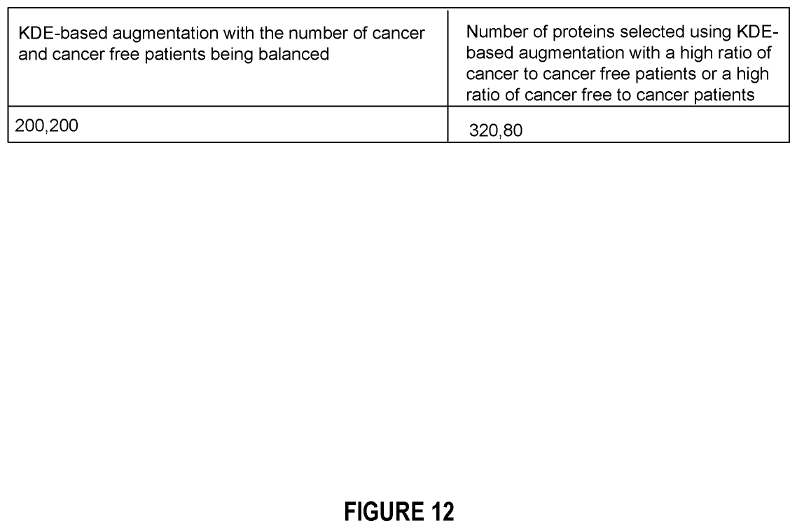

For analysis of the Blume et al dataset, a similar framework of example embodiments were used. The table of FIG. 12 shows the number of KDE-based samples used during training for distinguishing between lung cancer and cancer free patients.

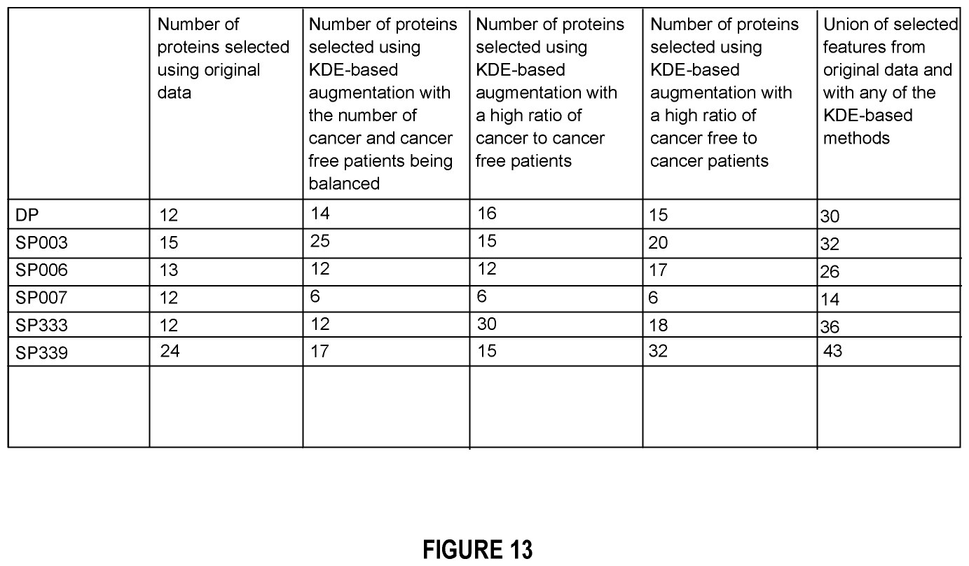

The training and validation steps of the example embodiment machine learning pipeline were applied using six different data inputs. One data input corresponded to a depleted plasma (DP) approach described in Blume et al. The other five data inputs represented protein intensities measured by five different nanoparticle ‘spions’ (SP003, SP006, SP007, SP333, SP339) described in Blume et al, each with different biophysical properties. The table of FIG. 13 shows the number of proteins selected using original data as well as each of the three KDE-based methods and the number of proteins in the union feature set.

The feature selection (including KDE-based methods) approach of the example embodiments enabled the following number of proteins to be selected: depleted plasma (DP): 30 out of an initial 419 proteins; SP003: 32 out of an initial 1238 proteins; SP006: 26 proteins out of an initial 1081 proteins; SP007: 14 out of an initial 897 proteins; SP333: 36 out of an initial 738 proteins; SP339: 43 out of an initial 897 proteins.

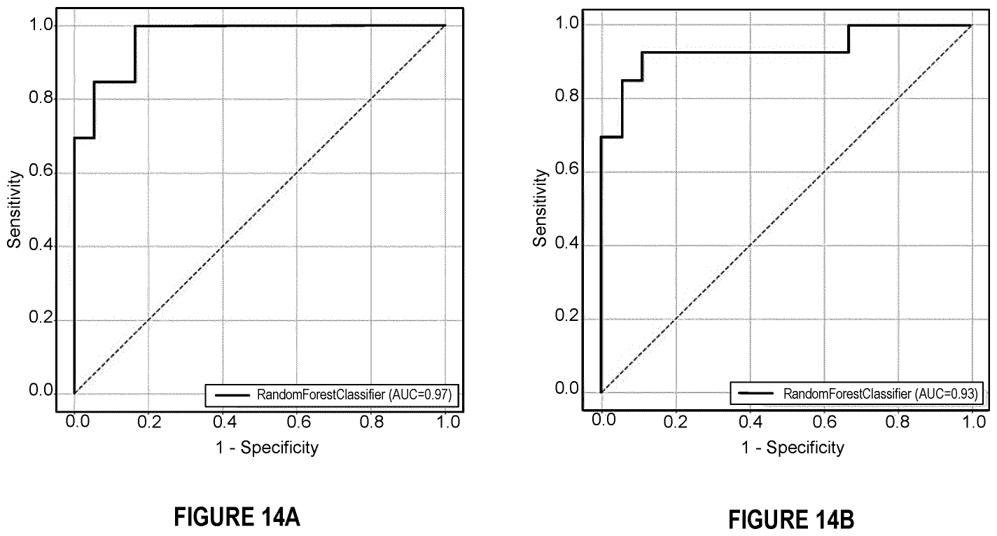

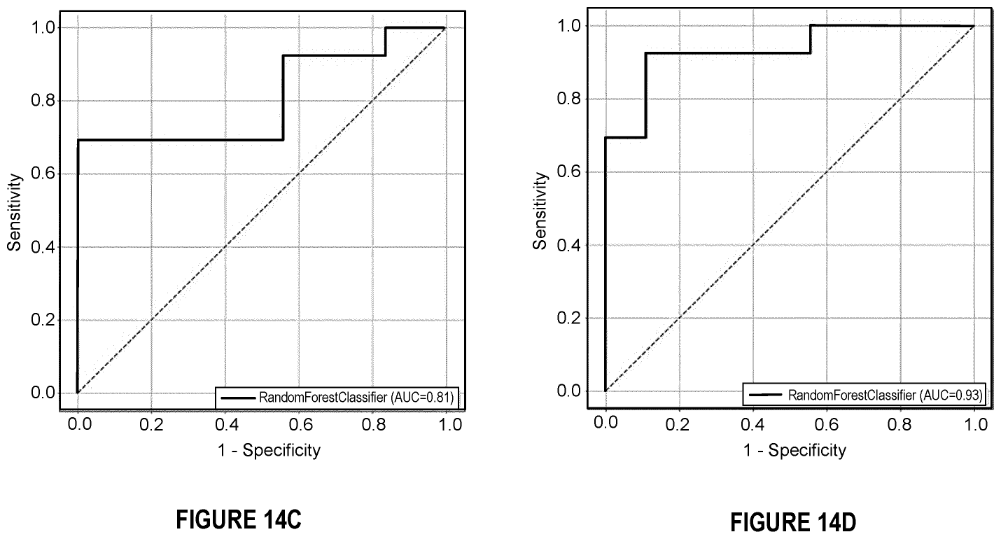

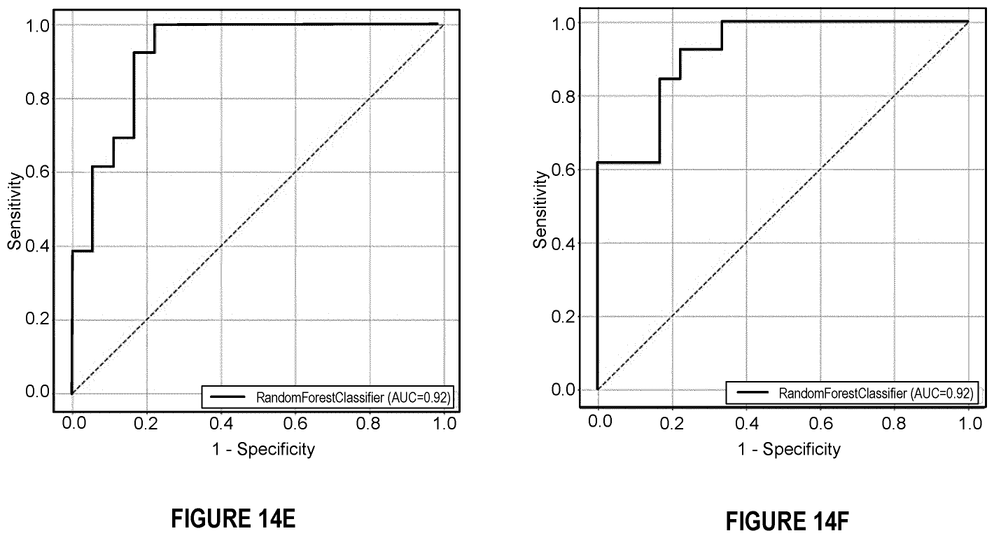

The best area under the receiver operating characteristic curve achieved by the example embodiments was 0.97 (see FIGS. 14A to 14F) compared to Blume et al's approach without the example embodiments in which a cross-validation performance of 0.91 area under the receiver operating characteristic curve was achieved for distinguishing between lung cancer and cancer free patients.

With reference to FIGS. 14A to 14F, for each spion or depleted plasma, the optimal subset of proteins used by the final classifier model to distinguish between cancer free and non-lung cancer samples is indicated (a) Depleted plasma in which the optimal protein set included 30 proteins (AUC 0.97); (b) SP003 in which the optimal protein set included 32 proteins (AUC 0.93); (c) SP006 in which the optimal protein set included 26 proteins (AUC 0.81); (d) SP007 in which the optimal protein set included 14 proteins (AUC 0.93); (e) SP333 in which the optimal protein set included 36 proteins (AUC 0.92); (f) SP339 in which the optimal protein set included 43 proteins (AUC 0.92).

In summary, the example embodiments enable rigorous feature selection, with selected features enabling cancer to be distinguished from cancer free patients whether there is a balanced or imbalanced ratio of cancer to cancer free patients. The example embodiments of the present disclosure enable superior performance of cancer detection compared to the gold standard approaches of other scientific groups without the example embodiments. Hence, the example embodiments appear well suited to clinical application.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

1. A computer implemented method for establishing training data for a machine learning classifier model for use in detecting a health abnormality in a liquid biopsy sample, the method comprising:
receiving a plurality of data sets, each data set comprising a plurality of features associated with a respective patient;
identifying all m training data sets of the plurality of data sets associated with a positive detection of the health abnormality and performing kernel density estimation on these data sets, followed by creating first ‘synthetic’ data sets consisting of p samples drawn at random from a positive health abnormality kernel density model;
identifying all n training data sets of the plurality of data sets associated with an absence of the health abnormality and performing kernel density estimation on these data sets, followed by creating second ‘synthetic’ datasets consisting of q samples drawn at random from an absent health abnormality kernel density model;
compiling training data comprising the first and second synthetic data sets;
identifying relevant features within respective first and second synthetic data sets of the training data, wherein a relevant feature, or combination of such features, provides a level of likelihood, above a threshold, of a positive indication of the health abnormality; and
optimizing training data via a removal of nonrelevant features.

2. The computer implemented method of claim 1, wherein the liquid biopsy comprises a sample of blood, urine, fecal matter, breath, or sputum.

3. The computer implemented method of claim 1, wherein the received data set comprises any one or more of DNA, epidemiology based data, proteomics, epigenetics, volatile organic molecules, metabolomics and/or microbiome based data.

4. The computer implemented method of claim 1, wherein the relevant features comprise biological features in a form of free molecules, exosomes, and/or apoptotic bodies and/or cells found in the liquid biopsy.

5. The computer implemented method of claim 1, further comprising: reformatting and normalizing the received data set.

6. The computer implemented method of claim 1, wherein the identifying relevant features further comprises performing a linear dimensionality reduction or non-linear dimensionality reduction techniques.

7. The computer implemented method of claim 1, wherein the identifying relevant features further comprises identifying relevant combinations of features via inputting the compiled training data into a variety of classifier types to identify non-linear feature interactions.

8. The computer implemented method of claim 1, wherein the identifying relevant features further comprises:
compiling randomized subsets of features, subsets containing x features;
inputting the randomized subsets into a plurality of different classifier models;
selecting feature subsets which enable the classifier of the plurality of different classifier models to yield a minimum predetermined metric in a validation set or else selecting a proportion of top-performing classifier feature subsets; and
for different z possible combinations of features which occur in at least a specified proportion of selected feature subsets, assigning a level of importance to each such combination based on an average decrease in classifier performance across all feature subsets comprising a particular feature combination, wherein relevant feature subsets of all features of the particular feature combination are deleted or features' values are permuted in relation to a response variable such that features of the particular combination are rendered non-informative.

9. The computer implemented method of claim 8, wherein the plurality of different classifier models comprise a learning classifier system.

10. The computer implemented method of claim 1, wherein the identifying relevant features further comprises:
inputting the compiled training data into a plurality of different learning classifier systems; and
assigning a level of importance to each feature combination which occurs in at least a specified proportion of selected feature subsets based on an average decrease in classifier performance across all feature subsets containing a particular feature combination, wherein relevant feature subsets of all features of the particular feature combination are deleted or the features' values are permuted in relation to a response variable such that the features of the particular combination are rendered non-informative.

11. A computer implemented method for selecting a machine learning classifier model for use in detecting a health abnormality in a liquid biopsy sample, the method comprising:
receiving a plurality of data sets, each data set comprising a plurality of features associated with a respective patient;
identifying all m training data sets of the plurality of data sets associated with a positive detection of the health abnormality and performing kernel density estimation on these data sets, followed by creating first ‘synthetic’ data sets consisting of p samples drawn at random from a positive health abnormality kernel density model;
identifying all n training data sets of the plurality of data sets associated with an absence of the health abnormality and performing kernel density estimation on these data sets, followed by creating second ‘synthetic’ datasets consisting of q samples drawn at random from an absent health abnormality kernel density model;
compiling training data comprising the first and second synthetic data sets;
identifying relevant features within respective first and second synthetic data sets of the training data, wherein a relevant feature, or combination of such features, provides a level of likelihood, above a threshold, of a positive indication of the health abnormality;
optimizing training data via a removal of nonrelevant features; and
training a plurality of different machine learning classifier models using the optimized training data.

12. The computer implemented method of claim 11, further comprising:
compiling a validation data set, wherein the validation data set comprises the identified relevant features, and wherein the validation data set is not equivalent to any data set comprised in the optimized training data set;
assessing a performance of the trained plurality of different machine learning classifier models on the validation data set; and
selecting a machine learning classifier, from the plurality of machine learning classifier models, wherein the selected machine learning classifier yields a percentage above a threshold of correctly detected health abnormalities.

13. The computer implemented method of claim 11, further comprising:
compiling k-folds, for k-fold validation;
assessing an average performance on validation folds in k-fold cross-validation; and
selecting a machine learning classifier, from the plurality of machine learning classifier models, wherein the selected machine learning classifier yields a percentage above a threshold of correctly detected health abnormalities.

14. The computer implemented method of claim 12, further comprising:
assessing a performance of the selected machine learning classifier on the validation data set via a receiver operating characteristic curve; and
optimizing parameters of the selected machine learning classifier to obtain predetermined sensitivity and selectivity ratios on the receiver operating characteristic curve.

15. The computer implemented method of claim 11, comprising:
receiving a test data set comprising the identified relevant features, wherein the test data set is not equivalent to any data set comprised in the optimized training data set, and wherein the test data set comprises data corresponding to at least one liquid biopsy sample;
assessing a performance of the selected machine learning classifier on the test data set; and
receiving an output of the selected machine learning classifier, wherein the output indicates a presence of the health abnormality in the liquid biopsy sample corresponding to the test data set.

16. The computer implemented method of claim 15, wherein the output is a probability or vote corresponding to a presence versus an absence of the health abnormality.

17. The computer implemented method of claim 1, wherein the classifier model is a one or more of a support vector machine, neural network, decision tree, random forest, boosted tree, logistic regression, lasso, k-nearest neighbor, and/or naïve byes.

18. The computer implemented method of claim 1, wherein the classifier model is a Michigan-style supervised learning classifier system or a Pittsburgh-style supervised learning system.

19. (canceled)

20. An analyzing unit comprising:
an input/output unit (301);
a memory (305); and
processing circuitry (303),
wherein the analyzing unit is configured to perform operations comprising:
receiving a plurality of data sets, each data set comprising a plurality of features associated with a respective patient;
identifying all m training data sets of the plurality of data sets associated with a positive detection of health abnormality and performing kernel density estimation on these data sets, followed by creating first ‘synthetic’ data sets consisting of p samples drawn at random from a positive health abnormality kernel density model;
identifying all n training data sets of the plurality of data sets associated with an absence of the health abnormality and performing kernel density estimation on these data sets, followed by creating second ‘synthetic’ datasets consisting of q samples drawn at random from an absent health abnormality kernel density model;
compiling training data comprising the first and second synthetic data sets;
identifying relevant features within respective first and second synthetic data sets of the training data, wherein a relevant feature, or combination of such features, provides a level of likelihood, above a threshold, of a positive indication of the health abnormality; and
optimizing training data via a removal of nonrelevant features.
